(19) United States
(12) Patent Application Publication
(10) Pub. No.: US 2024/0407725 A1
(43) Pub. Date: Dec. 12, 2024

(54) EARPHONE AND BIOMETRIC INFORMATION MEASUREMENT DEVICE

(71) Applicant: VIE, INC., Kanagawa (JP)

(72) Inventors: Yasuhiko IMAMURA, Kanagawa (JP); Yasushi NARUSE, Kanagawa (JP)

(21) Appl. No.: 18/699,709

(22) PCT Filed: Oct. 11, 2021
(86) PCT No.: PCT/JP2021/037521

§ 371 Date: Apr. 9, 2024

(51) Int. Cl. A61B 5/00

(52) U.S. Cl. CPC A61B 5/6817; A61B 5/7225; A61B 2560/045; A61B 2560/0462

(57) ABSTRACT

Biological signals are detected with higher precision. An earphone includes a first earpiece communicable with a communication terminal and worn in a right ear of a user, and a second earpiece communicable with the communication terminal and worn in a left ear of the user. The first earpiece includes a first sensor that acquires first biological information, a first time acquisition unit that acquires reference time information, and a first transmission unit that sends to the communication terminal, the first biological information associated with the reference time information. The second earpiece includes a second sensor that acquires second biological information, a second time acquisition unit that acquires reference time information, and a second transmission unit that sends, to the communication terminal, the second biological information associated with the reference time information.

### TECHNICAL FIELD

The present disclosure relates to an earphone and a biological information measurement device.

### BACKGROUND ART

Conventional earphones that acquire biological signals are known (see, for example, Patent Document 1 listed below).

### CITATION LIST

### Patent Document

Patent Document 1: Patent Publication JP-A-2014-215963

### SUMMARY

### Technical Problem

According to Patent Document 1, the electrodes that acquire biological signals are disposed in close proximity. Such close arrangement of the electrodes makes the potential difference of the biological signals, which are acquired by the respective electrodes, small, and causes the biological signals to cancel each other and to diminish.

An object of the present disclosure is to detect, with higher precision, biological signals.

### Solution to Problem

The present disclosure resides in an earphone, which includes a first earpiece communicable with a communication terminal and worn in a right ear of a user, and a second earpiece communicable with the communication terminal and worn in a left ear of the user. The first earpiece includes a first sensor that acquires first biological information, a first time acquisition unit that acquires reference time information, and a first transmission unit that sends, to the communication terminal, the first biological information associated with the reference time information. The second earpiece includes a second sensor that acquires second biological information, a second time acquisition unit that acquires reference time information, and a second transmission unit that sends, to the communication terminal, the second biological information associated with the reference time information.

### BRIEF DESCRIPTION OF DRAWINGS

FIG. 1 is a diagram for outlining the embodiment.

FIG. 2 is a block diagram illustrating a functional configuration of an earphone according to the embodiment.

FIG. 3 is a diagram illustrating an outer shape of the earphone according to the embodiment.

FIG. 4 is a cross-sectional view illustrating section IV-IV of FIG. 4.

### DESCRIPTION OF EMBODIMENTS

The embodiment is described below with reference to the accompanying drawings. For easier understanding of the description, the same constituent elements in the drawings are given the same reference numerals as much as possible and duplicate descriptions thereof will be omitted.

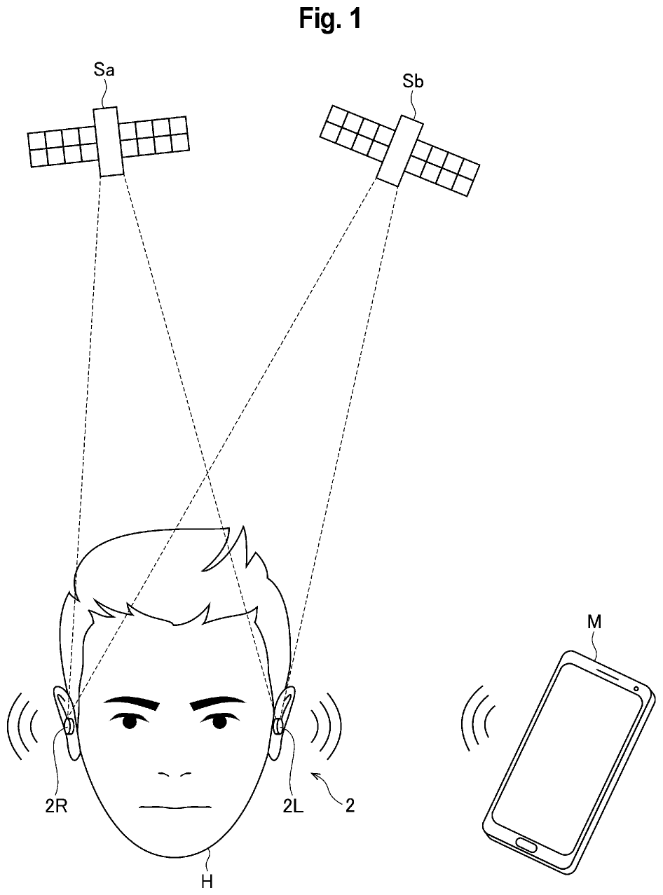

The embodiment will be outlined with reference to FIG. 1 Earphone 2 according to the embodiment includes a first earpiece 2R and a second earpiece 2L. The first earpiece 2R is worn in the right ear of a user H. The second earpiece 2L is worn in the left ear of the user H. The first earpiece 2R and second earpiece 2L are configured to be communicable with a smartphone M. The smartphone M is one example of a communication terminal. The first earpiece 2R and second earpiece 2L are configured to be able to receive GNSS signals from GNSS (Global Navigation Satellite System) satellites Sa and Sb.

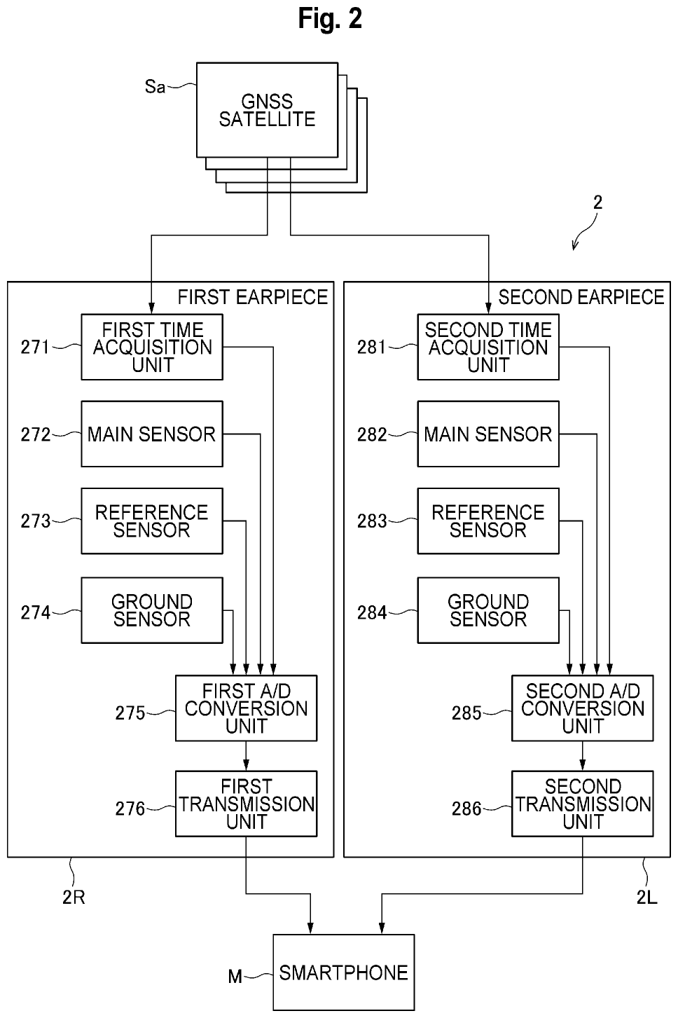

Next, the constituent elements of the earphone 2 according to this embodiment are described with reference to FIG. 2. The earphone 2 includes the first earpiece 2R and second earpiece 2L. The first earpiece 2R includes, as constituent elements thereof, a first time acquisition unit 271, a main sensor 272 (first sensor), a reference sensor 273 (third sensor), a ground sensor 274 (fifth sensor), a first A/D conversion unit 275, and a first transmission unit 276.

The first time acquisition unit 271 receives a GNSS signal sent from the GNSS satellite Sa and acquires absolute time information included in the GNSS signal. The first time acquisition unit 271 outputs the acquired absolute time information to the first A/D conversion unit 275. The first time acquisition unit 271 includes a GPS (Global Positioning System) chip, for example.

The main sensor 272 is provided at a position where it can acquire first biological information of the user H as an electrical signal. The installation position of the main sensor 272 will be described later. The main sensor 272 outputs the sensed first biological information to the first A/D conversion unit 275.

The reference sensor 273 is provided at a position where it can acquire third biological information of the user Has an electrical signal. The installation position of the reference sensor 273 will be described later. The reference sensor 273 outputs the sensed third biological information to the first A/D conversion unit 275.

The ground sensor 274 is a sensor that acquires ground potential information as an electrical signal. The installation position of the ground sensor 274 will be described later. The ground sensor 274 outputs the sensed ground potential information to the first A/D conversion unit 275.

The first A/D conversion unit 275 performs sampling on each information in accordance with a timing based on the absolute time information. Examples of sampling in accordance with a timing based on absolute time information include sampling the information according to the absolute time information every time, and sampling at regular timing, such as every 1 second, according to the absolute time information. The first A/D conversion unit 275 outputs the samples of each information to the first transmission unit 276.

The first transmission unit 276 sends the first biological information output from the main sensor 272 and the third biological information output from the reference sensor 273, each associated with absolute time information, to the communication terminal M. The first transmission unit 276 may generate first difference information that is a difference between the first biological information output from the main sensor 272 and the ground potential information output from the ground sensor 274, associate it with absolute time information, and transmit it to the communication terminal M. Similarly, the first transmission unit 276 may generate third difference information that is a difference between the third biological information output from the reference sensor 273 and the ground potential information output from the ground sensor 274, associate it with absolute time information, and transmit it to the communication terminal M. Associating each information with absolute time information (e.g., applying a time stamp) may be carried out in the first AD conversion unit 275.

In the example described above, the first transmission unit 276 sends the first biological information or the first difference information associated with absolute time information to the communication terminal M via a first channel. The absolute time information associated with the first biological information or first difference information is synchronized with the timing of the sensing by the main sensor 272. The first transmission unit 276 sends the third biological information or the third difference information associated with absolute time information to the communication terminal M via a third channel. The absolute time information associated with the third biological information or third difference information is synchronized with the timing of the sensing by the reference sensor 273. The third channel may be the same channel as the first channel, or a different channel. The biological information or ground potential information mentioned above each includes the information sampled by the first A/D conversion unit 275.

The second earpiece 2L includes, as constituent elements thereof, a second time acquisition unit 281, a main sensor 282 (second sensor), a reference sensor 283 (fourth sensor), a ground sensor 284 (sixth sensor), a second A/D conversion unit 285, and a second transmission unit 286.

The second time acquisition unit 281 receives a GNSS signal sent from the GNSS satellite Sa and acquires absolute time information included in the GNSS signal. The second time acquisition unit 281 outputs the acquired absolute time information to the second A/D conversion unit 285.

The main sensor 282 is provided at a position where it can acquire second biological information of the user H as an electrical signal. The installation position of the main sensor 282 will be described later. The main sensor 282 outputs the sensed second biological information to the second A/D conversion unit 285.

The reference sensor 283 is provided at a position where it can acquire fourth biological information of the user H as an electrical signal. The installation position of the reference sensor 283 will be described later. The reference sensor 283 outputs the sensed fourth biological information to the second A/D conversion unit 285.

The ground sensor 284 is a sensor that acquires ground potential information as an electrical signal. The installation position of the ground sensor 284 will be described later. The ground sensor 284 outputs the sensed ground potential information to the second A/D conversion unit 285.

The second A/D conversion unit 285 performs sampling on each information in accordance with a timing based on the absolute time information. Examples of sampling in accordance with a timing based on absolute time information include sampling the information according to the absolute time information every time, and sampling at regular timing, such as every 1 second, according to the absolute time information. The second A/D conversion unit 285 outputs the samples of each information to the second transmission unit 286.

The second transmission unit 286 sends the second biological information output from the main sensor 282 and the fourth biological information output from the reference sensor 283, each associated with absolute time information, to the communication terminal M. The second transmission unit 286 may generate second difference information that is a difference between the second biological information output from the main sensor 282 and the ground potential information output from the ground sensor 284, associate it with absolute time information, and transmit it to the communication terminal M. Similarly, the second transmission unit 286 may generate fourth difference information that is a difference between the fourth biological information output from the reference sensor 283 and the ground potential information output from the ground sensor 284, associate it with absolute time information, and transmit it to the communication terminal M. Associating each information with absolute time information (e.g., applying a time stamp) may be carried out in the second AD conversion unit 285.

In the example described above, the second transmission unit 286 sends the second biological information or the second difference information associated with absolute time information to the communication terminal M via a second channel. The absolute time information associated with the second biological information or second difference information is synchronized with the timing of the sensing by the main sensor 282. The second transmission unit 286 sends the fourth biological information or the fourth difference information associated with absolute time information to the communication terminal M via a fourth channel. The absolute time information associated with the fourth biological information or fourth difference information is synchronized with the timing of the sensing by the reference sensor 283. The fourth channel may be the same channel as the second channel, or a different channel. The biological information or ground potential information mentioned above each includes the information sampled by the second A/D conversion unit 285.

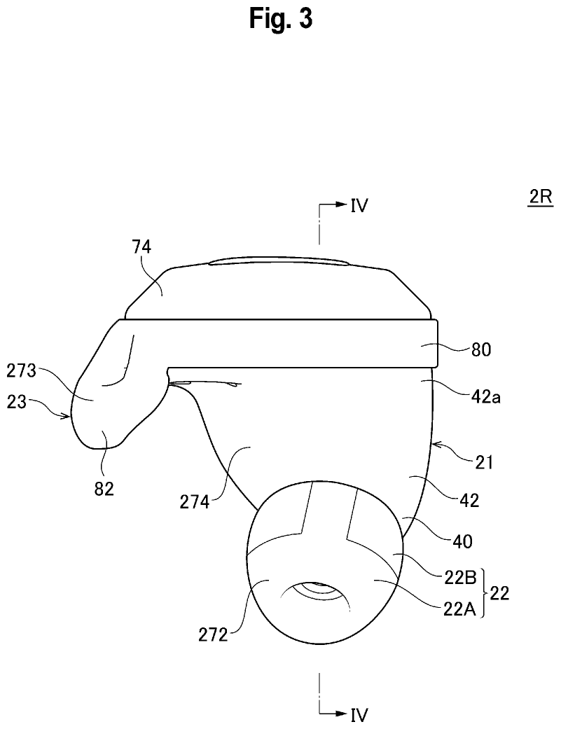

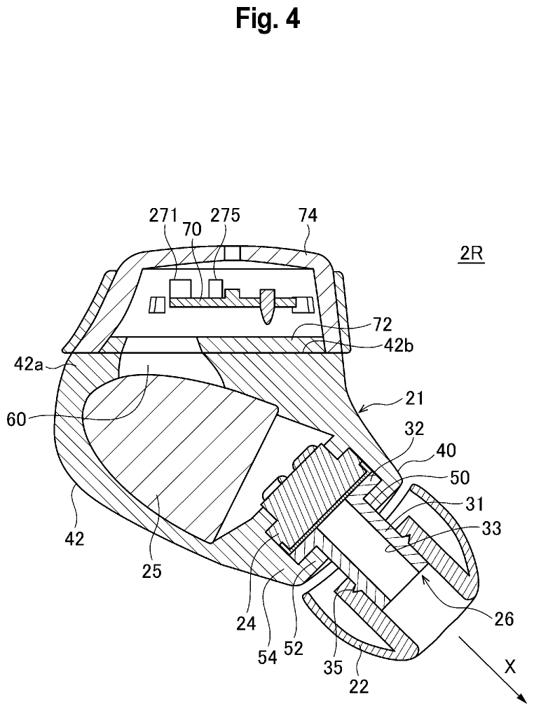

Next, the outer shape of the earpieces and how each of the sensors is disposed in one example will be described with reference to FIG. 3 and FIG. 4. FIG. 3 is a cross-sectional view illustrating section IV-IV of FIG. 4. The first earpiece 2R and second earpiece 2L are basically configured the same except that they are different in their shape, respectively suited to be worn in right and left ears. Therefore, the first earpiece 2R will be described as one example.

The first earpiece 2R includes a housing 21, an ear tip 22, and a wing 23. The housing 21 is a component that has a cavity inside; this cavity accommodates a speaker 24 and a battery 25. The ear tip 22 is attached to a nozzle 26 protruding from the housing 21.

The nozzle 26 has a shape in which a flange is formed at one end of a tubular member. Specifically, the nozzle 26 includes a tubular part 31 that is positioned in the external auditory canal of the wearer when the first earpiece 2R is worn, a flange part 32 fixed to the housing 21, and a sound conduction part 33 that connects the respective interiors of the tubular part 31 and flange part 32 in a first direction X.

The tubular part 31 extends in the first direction X such as to protrude from the housing 21. At the distal end of the tubular part 31 is formed an engagement protrusion 35 for removable engagement of the ear tip 22. The flange part 32 is formed at the base end of the tubular part 31. The sound conduction part 33 functions as a passage for the sound from the speaker 24. This nozzle 26 is formed as a rigid member. As long as the nozzle 26 has this characteristic, the material forming the nozzle shall not be limited; hard ABS resin can be named as one example.

The housing 21 includes a nozzle fixing part 40 that secures the flange part 32 of the nozzle 26, and an extended part 42 extending from the nozzle fixing part 40 in the opposite direction from the nozzle 26 and wider than the nozzle fixing part 40. The nozzle fixing part 40 includes an opening 50 for the tubular part 31 of the nozzle 26 to pass through, a restriction part 52 that makes contact with the flange part 32 of the nozzle 26 to stop detachment of the nozzle 26 from the opening 50, and an encircling part 54 that is a portion joining the restriction part 52 and the extended part 42 and surrounding the flange part 32. The nozzle 26 may be fixed in the nozzle fixing part 40 by fitting the flange part 32 into the encircling part 54, for example, or, by bonding the flange part 32 either to the restriction part 52 or the encircling part 54.

The extended part 42, as a whole, is shaped such as to gradually become wider away from the nozzle fixing part 40. The extended part 42 accommodates therein the speaker 24 on the side closer to the nozzle 26, and the battery 25 in a portion that is even wider than this part accommodating the speaker 24. The extended part 42 further has an opening 60 for wiring at an end 42a where the widening ends. The wired connection of the speaker 24 and battery 25 to a substrate 70 is achieved via this opening 60. The opening 60 is also used when inserting the speaker 24 and battery 25 into the housing 21 during the assembly of the first earpiece 2R.

The end face 42b of the extended part 42 where the widening ends is formed as a flat surface. On the end face 42b is placed a plate 72 to which the substrate 70 is fixed, and is mounted a cover 74 that covers the substrate 70 and the plate 72. An antenna is provided to the substrate 70 for wireless communication. The antenna is compatible with a wireless communication standard such as Bluetooth (Registered Trademark), for example. Accordingly, the first earpiece 2R is configured as a wireless earphone; it is connected wirelessly to devices such as a mobile terminal or laptop PC, and exchanges data such as sound with these devices. The first time acquisition unit 271 and first transmission unit 276 are provided to the substrate 70.

The wing 23 includes an annular mounting part 80 that is fitted around the circumference of the cover 74, and a wingtip 82 protruded from the mounting part 80.

The wingtip 82 protrudes toward the ear tip 22 from the mounting part 80 to substantially form a U shape. The wingtip 82 mainly functions as a hook held on the outer ear of the wearer when the first earpiece 2R is worn, providing a support to prevent the first earpiece 2R from coming off of the wearer's concha. The wing 23 may be made of a material having elasticity and flexibility, similarly to the housing 21.

The ear tip 22 is composed of a first member 22A that has conductivity, and a second member 22B. The first member 22A and second member 22B are made of different materials, and separately removable, for example. The shape of the first member 22A is not limited to the example shown in FIG. 3 and FIG. 4; the first member 22A may have any design as long as it includes a portion that makes contact with an inner wall of the wearer's external auditory canal, with this portion making suitable contact with the external auditory canal. Preferably, this contact portion should have a large surface area.

The ear tip 22 includes the first member 22A positioned closer to the eardrum, and the second member 22B positioned closer to the housing 21. The first member 22A is fabricated using conductive rubber, for example, this conductive rubber containing silver or silver chloride. Preferably, to ensure a suitable level of conductivity, the content of silver or silver chloride should be a predetermined mass % or more of the conductive substance contained in the conductive rubber.

The first member 22A may be made of a silicone material containing metal filler. A material having high conductivity for the first member 22A can be made by mixing any of silver, copper, gold, aluminum, zinc, and nickel as metal filler with a suitable proportion. The filler to be contained need not necessarily be silver or silver chloride entirely; the filler may partly be silver or silver chloride. Namely, the content of the silver or silver chloride can be reduced, which allows the hardness of the rubber to be lowered, and a conductive rubber with a suitable hardness to be made.

The second member 22B is preferably made of an inexpensive non-conductive elastic material (such as, for example, silicone rubber).

The housing 21 is fabricated using conductive rubber, for example, this conductive rubber containing silver or silver chloride. Preferably, to ensure a suitable level of conductivity, the content of silver or silver chloride should be a predetermined mass % or more of the conductive substance contained in the conductive rubber.

The housing 21 may be made of a silicone material containing metal filler. A material having high conductivity for the first member 22A can be made by mixing any of silver, copper, gold, aluminum, zinc, and nickel as metal filler with a suitable proportion. The filler to be contained need not necessarily be silver or silver chloride entirely; the filler may partly be silver or silver chloride. Namely, the content of the silver or silver chloride can be reduced, which allows the hardness of the rubber to be lowered, and a conductive rubber with a suitable hardness to be made.

The wingtip 82 is fabricated using conductive rubber, for example, this conductive rubber containing silver or silver chloride. Preferably, to ensure a suitable level of conductivity, the content of silver or silver chloride should be a predetermined mass % or more of the conductive substance contained in the conductive rubber.

The wingtip 82 may be made of a silicone material containing metal filler. A material having high conductivity for the first member 22A can be made by mixing any of silver, copper, gold, aluminum, zinc, and nickel as metal filler with a suitable proportion. The filler to be contained need not necessarily be silver or silver chloride entirely; the filler may partly be silver or silver chloride. Namely, the content of the silver or silver chloride can be reduced, which allows the hardness of the rubber to be lowered, and a conductive rubber with a suitable hardness to be made.

Being configured as described above, the first member 22A of the ear tip 22, the housing 21, and the wingtip 82 are conductive. While the first member 22A is used as the main sensor 272, the housing 21 as the ground sensor 274, and the wingtip 82 as the reference sensor 273 in the example shown in this embodiment, this example is not limiting. The conductive material of the housing 21, the conductive material of the first member 22A, and the conductive material of the wingtip 82, may be the same, or different from each other. The conductivity of the first member 22A may be made higher than the conductivity of the housing 21 and the wingtip 82.

As described above, the earphone 2 according to this embodiment includes the first earpiece 2R communicable with a communication terminal and worn in the right ear of the user H, and the second earpiece 2L communicable with the communication terminal and worn in the left ear of the user H. The first earpiece 2R includes the main sensor 272, which is one example of a first sensor that acquires first biological information, the first time acquisition unit 271 that acquires absolute time information that is one example of reference time information, and the first transmission unit 276 that sends the first biological information associated with the absolute time information to the communication terminal. The second earpiece 2L includes the main sensor 282, which is one example of a second sensor that acquires second biological information, the second time acquisition unit 281 that acquires absolute time information that is one example of reference time information, and the second transmission unit 286 that sends the second biological information associated with the absolute time information to the communication terminal.

The first transmission unit 276 sends the first biological information associated with absolute time information to the communication terminal from the right ear in which the first earpiece 2R is worn, so that the communication terminal is able to accurately determine at which time the biological information was acquired. The second transmission unit 286 sends the second biological information associated with absolute time information to the communication terminal from the left ear in which the second earpiece 2L is worn, so that the communication terminal is able to accurately determine at which time the biological information was acquired. Earpieces are worn in ears; plural sensors for acquiring biological information provided inside the same earpiece generate signals with a small potential difference. Such signals cancel each other, making it hard to increase the precision of the acquired biological information. Providing sensors separately in different earpieces, with each sensor acquiring biological information, can resolve the issue that a sufficient potential difference cannot be achieved. However, processing the biological information acquired in each of different earpieces and sent to a communication terminal may lead to another issue that communication delays or the like may cause errors. In this embodiment, the biological information acquired in each earpiece is each associated with absolute time information and sent to the communication terminal, which resolves the issue of possible errors caused by communication delays, and enables acquisition of accurate biological information with a sufficient potential difference.

The absolute time information included in a GNSS signal is used as one example of reference time information in this embodiment. Other types of time information may be used as the reference time information as long as the information is precise enough to allow the time to be specified at which the biological information was acquired in each earpiece. For example, the first time acquisition unit 271 and second time acquisition unit 281 may acquire signals that provide the respective earpieces with reference times with a tolerance of 1 ms or less. The reference time information not only indicates the time at which the biological information was acquired but can also be used as the information for synchronizing the biological information with the biological information acquired by the other earpiece. The reference time information may also be substituted by the synchronizing information for synchronizing the biological information with the biological information acquired by the other earpiece.

As described above, the first earpiece 2R includes the reference sensor 273, one example of a third sensor, which acquires third biological information at a position different from that of the main sensor 272 that is one example of the first sensor. The first transmission unit 276 sends the first biological information and the third biological information, each associated with absolute time information that is one example of reference time information, to the communication terminal. The second earpiece 2L includes the reference sensor 283, one example of a fourth sensor, which acquires fourth biological information at a position different from that of the main sensor 282 that is one example of the second sensor. The second transmission unit 286 sends the second biological information and the fourth biological information, each associated with absolute time information that is one example of reference time information, to the communication terminal.

This way, biological information acquired at two different positions is associated with absolute time information and sent to the communication terminal from the two earpieces, so that the communication terminal can acquire four sets of biological information, with the absolute time information providing accurate records of the times of acquisition. For example, the first biological information and fourth biological information can be processed as main biological information and reference biological information, respectively, and cross-referenced, or the second biological information and third biological information can be processed as main biological information and reference biological information, respectively, and cross-referenced. Such cross-reference processing can augment a signal component, or increase a potential difference.

As described above, the first time acquisition unit 271 receives a GNSS signal sent from the GNSS satellite Sa and acquires reference time information including absolute time information. The second time acquisition unit 281 receives a GNSS signal sent from the GNSS satellite Sa and acquires reference time information including absolute time information.

As described above, the first earpiece 2R includes the ground sensor 274, which is one example of a fifth sensor that acquires ground potential information at a different position from that of the main sensor 272 that is one example of the first sensor and that of the reference sensor 273 that is one example of the third sensor. The first transmission unit 276 sends first difference information between the first biological information and the ground potential information, and second difference information between the third biological information and the ground potential information, each associated with reference time information, to the communication terminal. The second earpiece 2L includes the ground sensor 284, which is one example of a sixth sensor that acquires ground potential information at a different position from that of the main sensor 282 that is one example of the second sensor and that of the reference sensor 283 that is one example of the fourth sensor. The second transmission unit 286 sends third difference information between the second biological information and the ground potential information, and fourth difference information between the fourth biological information and the ground potential information, each associated with reference time information, to the communication terminal. The third difference information may be a calculated difference between the first biological information and the ground potential information sampled by the first A/D conversion unit 275 at the same timing. The fourth difference information may also be calculated by the second A/D conversion unit 285 similarly to the third difference information.

The difference information, which is a difference between a potential indicative of biological information and the ground potential, sent to the communication terminal in association with reference time information, allows for acquisition of biological information with higher signal precision, in association with reference time.

As described above, the first earpiece 2R has the first A/D conversion unit 275 that samples the first electrical signal measured by the main sensor 272 that is one example of the first sensor, based on the reference time information. The first transmission unit 276 may send the first biological information sampled by the first A/D conversion unit 275 and associated with the reference time information to the communication terminal. The second earpiece 2L has the second A/D conversion unit 285 that samples the second electrical signal measured by the main sensor 282 that is one example of the second sensor, based on reference time information. The second transmission unit 286 may send the second biological information sampled by the second A/D conversion unit 285 and associated with the reference time information to the communication terminal. This allows the sampling timing of the left and right earpieces to be matched so that the biological information from the left and right earpieces can be synchronized better. For example, even when each biological information is given an accurate time stamp, the biological information from the left and right earpieces can hardly be synchronized correctly if the sampling timings of the left and right sensors are not in sync. This issue is resolved by matching the sampling timings of the left and right sensors based on reference time information. The sampling timings of the third biological information, fourth biological information, and respective sets of ground potential information may also be matched based on reference time information.

While brain wave information acquired from the external auditory canal is used to estimate a wider range of brain information in the embodiment described above, the information to be predicted is not limited to a wider range of brain wave information. For example, various pieces of biological information that can be acquired from a human body can be estimated from the brain wave information or biological information that can be acquired from the external auditory canal.

Specific examples of other biological signals than brain waves that can be acquired from around the head include electrooculogram signals, heart rate signals acquired over the carotid artery or the like, and myopotential signals acquired over the masseter muscle or temporal muscle. ECG signals, for example, are also an example of a biological signal obtainable from a human body. These various pieces of biological information may be measured using respective biological sensors dedicated to measure the corresponding biological information.

While an earphone having a pair of earpieces was described as one example of a biological information acquiring device in the embodiment above, the technique of the present disclosure can be applied also to at least two biological information measurement devices that measure biological information at different positions. For example, one of these devices may be one of the earpieces according to the present disclosure, and the other device may be eyewear (glasses), for example, which has a configuration similar to that of the other one of the earpieces according to the present disclosure.

According to the above example, the biological information measurement device includes a first device communicable with a communication terminal and worn in a first predetermined portion of a user, and a second device communicable with the communication terminal and worn in a second predetermined portion of the user. The first device includes a first sensor that acquires first biological information, a first time acquisition unit that acquires reference time information, and a first transmission unit that sends the first biological information associated with reference time information to a communication terminal.

The second device includes a second sensor that acquires second biological information, a second time acquisition unit that acquires reference time information, and a second transmission unit that sends the second biological information associated with reference time information to the communication terminal. This enables cross-reference signal processing of biological information between two devices utilizing absolute time synchronization techniques. The configurations and processing in the embodiment described in the foregoing can be applied to this biological information measurement device.

The embodiment has been described with reference to specific examples above. However, the present disclosure is not limited to these specific examples. As long as the features of the present disclosure are entailed, any variations with design changes made to these specific examples as appropriate by those skilled in the art are also included in the scope of the present disclosure. Various elements in the specific examples described above, arrangements, conditions, shapes and so on are not limited to the illustrated examples and may be changed as required. The elements in the specific examples described above may be variously combined as suited as long as there is no technical inconsistency.

### REFERENCE SIGNS LIST

2 Earphone
2R First earpiece
2L Second earpiece
21 Housing
22 Ear tip
23 Wing
24 Speaker
25 Battery
26 Nozzle
42 Extended part
60 Opening
70 Substrate
272, 282 Main sensor
273, 283 Reference sensor
274, 274 Ground sensor
Sa, Sb Satellite

1. An earphone comprising:
a first earpiece communicable with a communication terminal and worn in a right ear of a user; and
a second earpiece communicable with the communication terminal and worn in a left ear of the user,
the first earpiece including a first sensor that acquires first biological information, a first time acquisition unit that acquires reference time information, and a first transmission unit that sends, to the communication terminal, the first biological information associated with the reference time information,
the second earpiece including a second sensor that acquires second biological information, a second time acquisition unit that acquires reference time information, and a second transmission unit that sends, to the communication terminal, the second biological information associated with the reference time information.

2. The earphone according to claim 1, wherein
the first earpiece includes a third sensor that acquires third biological information at a position different from that of the first sensor,
the first transmission unit sends, to the communication terminal, the first biological information and the third biological information, each associated with the reference time information,
the second earpiece includes a fourth sensor that acquires fourth biological information at a position different from that of the second sensor, and
the second transmission unit sends, to the communication terminal, the second biological information and the fourth biological information, each associated with the reference time information.

3. The earphone according to claim 1, wherein
the first time acquisition unit receives a GNSS signal sent from a GNSS satellite and acquires the reference time information including absolute time information, and
the second time acquisition unit receives a GNSS signal sent from a GNSS satellite and acquires the reference time information including absolute time information.

4. The earphone according to claim 2, wherein
the first earpiece includes a fifth sensor that acquires ground potential information at a position different from that of the first sensor and that of the third sensor,
the first transmission unit sends, to the communication terminal, first difference information between the first biological information and the ground potential information, and second difference information between the third biological information and the ground potential information, each associated with the reference time information,
the second earpiece includes a sixth sensor that acquires ground potential information at a position different from that of the second sensor and that of the fourth sensor, and
the second transmission unit sends, to the communication terminal, third difference information between the second biological information and the ground potential information, and fourth difference information between the fourth biological information and the ground potential information, each associated with the reference time information.

5. The earphone according to claim 1, wherein
the first earpiece includes a first A/D conversion unit that samples a signal measured by the first sensor, based on the reference time information,
the first transmission unit sends, to the communication terminal, the first biological information sampled by the first A/D conversion unit and associated with the reference time information,
the second earpiece includes a second A/D conversion unit that samples a signal measured by the second sensor, based on the reference time information, and
the second transmission unit sends, to the communication terminal, the second biological information sampled by the second A/D conversion unit and associated with the reference time information.

6. A biological information measurement device, comprising:
a first device communicable with a communication terminal and worn in a first predetermined portion of a user; and
a second device communicable with the communication terminal and worn in a second predetermined portion of the user,
the first device including a first sensor that acquires first biological information, a first time acquisition unit that acquires reference time information, and a first transmission unit that sends, to the communication terminal, the first biological information associated with the reference time information, and
the second device including a second sensor that acquires second biological information, a second time acquisition unit that acquires reference time information, and a second transmission unit that sends, to the communication terminal, the second biological information associated with the reference time information.
